(19) United States
(12) Patent Application Publication
(10) Pub. No.: US 2022/0378978 A1
(43) Pub. Date: Dec. 1, 2022

(54) ZIRCONIUM-COATED IMPLANT COMPONENT AND USE OF SAME

(71) Applicant: WALDEMAR LINK GMBH & CO. KG, Hamburg (DE)

(72) Inventors: Richard CSASZAR, Bad Segeberg (DE); Helmut D. LINK, Hamburg (DE)

(73) Assignee: WALDEMAR LINK GMBH & CO. KG, Hamburg (DE)

(21) Appl. No.: 17/755,719

(22) PCT Filed: Nov. 13, 2020
(86) PCT No.: PCT/EP2020/082135

§ 371 Date: May 6, 2022

(30) Foreign Application Priority Data
Nov. 13, 2019 (EP) 19208824.3

(51) Int. Cl. A61L 27/30; A61L 27/04; A61F 2/30; A61L 27/50

(52) U.S. Cl. CPC A61L 27/306; A61L 27/047; A61F 2/3094; A61L 27/50; A61L 2420/02; A61L 2430/24; A61F 2002/30332; A61F 2002/3065; A61F 2002/30604; A61F 2002/30971; A61L 2400/18; A61F 2310/00029; A61F 2310/00023; A61F 2310/00485

(57) ABSTRACT

The present disclosure relates to an implant component (10, 20) having at least one connecting portion (30, 60), the connecting portion being at least partly coated with a Zr coating and the coating having a thickness of 1-20 μm, preferably 1-6 μm. The present disclosure further relates to a modular endoprosthesis comprising an implant component, to the use of a Zr coating to prevent crevice corrosion and/or fretting corrosion, and to the use of an implant component in patients suffering from a metal allergy.

### TECHNICAL FIELD

The present disclosure relates to an implant component having a connecting portion for connecting to a further implant component, and relates to an implant having at least one implant component. The present disclosure also relates to the use of an implant component or an implant.

### PRIOR ART

Endoprostheses, for example hip endoprostheses, can have a modular structure, i.e. they are made up of at least two components that can be selected separately from one another. This structure gives the prostheses a high degree of adaptability to the individual requirements of a patient.

The individual requirements of a patient can include, for example, specific dimensions, geometries, material combinations, and/or fastening mechanisms. The use of modular prostheses allows the multitude of requirements to be taken into account without having to stock or individually manufacture implants for every possible permutation.

Individual implant components of a modular endoprosthesis can be assembled using connecting portions to form an endoprosthesis or an implant. Cone connections in particular have proven their value in these cases. For example, a cone connection can have a substantially conical projection (male cone) and an opening with a tapering section (female cone). The male cone and female cone each have a cone axis that coincides when the projection is inserted into the opening (i.e., when the implant components are connected). The cone connection is therefore also self-centering.

When the male cone is introduced into the female cone and a force is applied to the implant components in the direction of the cone axis, this leads to an elastic, radially-oriented expansion of the female cone with respect to the cone axis and to a compression of the male cone. The recovery forces resulting from expansion and compression in turn generate a clamping force which, during self-locking, leads to frictional connection of the implant components.

Such cone connections are provided, for example, in hip endoprostheses. Simple modular hip endoprostheses have such a connection, for example between a prosthesis shaft and a femoral head (joint head). Furthermore, a number of modular hip endoprostheses are known which have an intermediate piece arranged between the prosthesis shaft and the femoral head in order to facilitate better adaptation to the anatomy of the patient. In this case, for example, the prosthesis shaft and the intermediate piece and/or the intermediate piece and the femoral head can be connected to a cone connection as described above.

However, it has been shown that with the modular endoprostheses described above, crevice corrosion and/or friction corrosion (technical term: fretting or fretting corrosion) can occur in the cone connection. In particular, this phenomenon affects the contact surfaces of the implant components, that is, the lateral surface of the male cone and the inner circumferential surface of the female cone.

Such corrosion phenomena can sometimes lead to fatal failure of the implant. This failure occurs in particular as the male cone breaking off. Micro-movements between the connecting surfaces of two implant components and the resulting stresses are sometimes found to be responsible for the corrosion. The corrosion can sometimes manifest in the form of cracks and/or abrasion of a passive layer on the surface of the implant material. It is noted that corrosion phenomena can occur not only in metal-metal pairings, but also in metal-ceramic pairings, such as, for example, hip endoprostheses having a metal shaft and ceramic head.

Furthermore, it is assumed that such cone connections function according to the principle described above, but that the connection does not take place over the entire length of the cone due to production reasons. For instance, it was found that the cone connection is primarily established in the proximal area, i.e. at the end of the tapering cone. The result is higher stresses on its surface, these stresses promoting the corrosion phenomena described above. This effect is also amplified by the fact that cone connections are generally produced by machining, in particular by turning.

This creates a characteristic wavy surface in the micrometer range. This can also cause stress peaks and local deformations in a cone connection.

In addition to the material failure described above, the corrosion phenomena described above also regularly lead to the release of metal ions, metal oxides, metal organophosphates, and/or small metal particles, which in turn increase mechanical abrasion phenomena. As a result, patients may experience pain, aseptic loosening of the endoprosthesis, and/or negative consequences for the surrounding tissue. Thus, this can lead in particular to metallosis, i.e. an unnatural occurrence of wear particles in the tissue. One possible consequence of this is the development of so-called pseudotumors. Allergic reactions are also possible and can also necessitate prosthesis revision.

In the past, structural and production measures have sometimes been taken in order to counteract the problems described above. For example, attempts were made to reduce the degree of corrosion phenomena using more precise manufacturing processes or using adapted contact surface geometries. DE 102014206151 A1 also discloses a cone connection having a TiNb coating, which is less susceptible to corrosion than uncoated connections.

Another option considered was the use of nitride coatings to improve the wear properties of implant components. These are used, for example, for the running surfaces of endoprostheses. However, it has been found that different wear conditions than in a connecting portion prevail there. This may be one reason that nitride coatings have not led to satisfactory results here.

Since neither premature failure nor interactions with abrasion material can be ruled out in connecting portions with or without a coating, efforts to further reduce the corrosion phenomena beyond the level achieved so far are still ongoing.

### SUMMARY OF THE INVENTION

The underlying object of the present disclosure was to counteract the problems described above and to bring about improvements in an implant component having a connecting portion and in particular improvements with regard to crevice corrosion and/or friction corrosion (fretting corrosion). This object is achieved by an implant component according to claim 1, a modular endoprosthesis according to claim 10, use according to claim 13, and use according to claim 14. Preferred embodiments are specified in the subordinate claims.

An implant component according to the present disclosure has at least one connecting portion which is at least partly coated with a Zr coating. Preferably, the entire connecting portion is coated with a Zr coating. It is particularly preferred that at least those surfaces of the connecting portion that are designed to come into contact with surfaces of a connecting portion counterpart of another implant component are coated with a Zr coating.

The Zr coating has a thickness between 1 μm and 20 μm, preferably between 1 μm and 6 μm. In the context of the present disclosure, Zr is elemental zirconium (element with atomic number 40 in the periodic table), but not zircon (Zr[SiO₄]) or other zirconium-based minerals or ceramics. According to the present disclosure, zirconium-based oxides that can form on the surface of a Zr coating in the form of a passive layer are associated with a Zr coating.

The implant component described above can be associated with significantly reduced susceptibility to crevice corrosion and/or friction corrosion in the region of the connecting portion compared to the prior art. This property is largely due to the selection of the coating material (Zr) in combination with the layer thickness.

With the measurement method described below, it was shown that a connecting portion having a Zr coating (with a layer thickness according to the disclosure) is significantly less susceptible to crevice corrosion and/or friction corrosion (fretting corrosion) than, for example, a connecting portion with titanium-niobium (TiNb) or a connecting portion embodied with a cobalt base alloy (e.g., CoCrMo).

Due to the lower susceptibility to crevice corrosion and/or friction corrosion (fretting corrosion), it is assumed that the passive layer on the surface of the Zr coating is significantly more stable than the passive layer of other metallic coatings or materials. In addition, previous studies indicate a high degree of biocompatibility, which in particular prevents allergic reactions.

Furthermore, it is assumed that the low susceptibility of the Zr coating to corrosion is due to the mechanical properties of this coating. In particular, the coating has sufficient ductility. As a result, more uniform contact at the connecting portion is enabled, and thus there is a reduction in stress peaks. This is an advantage, for example, in the case of the above-mentioned, production-related wavy surface structure.

The layer thickness of the Zr coating of an implant component according to the present disclosure is preferably between 3 μm and 6 μm, and particularly preferably between 3 μm and 5 μm.

The aim is to select the thickness of the layer to be high enough that a coating that withstands the mechanical loads is achieved. At the same time, it is desirable not to choose a coating that is too thick, so that a stable frictional connection is achieved between the surfaces of the connection region.

Producing a Zr layer having a large thickness is associated with increased costs. On the other hand, if the layer is too thin, the desired effect (reducing susceptibility to crevice corrosion and/or friction corrosion) may not be permanently and reproducibly achieved. The (particularly) preferred layer thicknesses listed above are associated with maintainable low production costs and at the same time make it possible to reduce the susceptibility of the connecting portion to crevice corrosion and/or friction corrosion compared to known coatings. The layer thicknesses mentioned above can therefore also be considered a solution to a multidimensional optimization problem with regard to production costs and the occurrence of the desired effect.

An implant component according to the present disclosure may have a connecting portion embodied with a female and/or male cone. The female and/or male cone is preferably rotationally symmetrical.

A cone connection can be considered self-stabilizing due to its wedging properties. In addition, it is characterized by relatively low production costs and allows precise production of the mating surfaces (for example, with a concentricity tolerance of less than 0.1 mm, less than 0.05 mm, or less than 0.01 mm). As a result of the high manufacturing precision that can be achieved with such geometries, the amount of free volume between mating surfaces can be reduced to a minimum. Because of this, the forces transmitted via the cone connection are evenly distributed. Stress peaks are prevented.

If the cone connection is embodied rotationally symmetrical, the operating surgeon has a greater degree of freedom for aligning the implant when it is being inserted, specifically a degree of freedom of rotation about the cone axis. With certain implants, this degree of freedom of rotation allows the implant geometry to be quickly and easily adapted to the patient geometry.

As an alternative to the cone connection, the connecting portion can be designed, for example, as an axis-parallel cylinder and/or as an axis-parallel bore. If the connecting portion is connected to another implant component, the axis-parallel cylinder and/or the axis-parallel bore can be part of a fitting system, for example with a press fit or a transition fit (e.g. H7p6 or H7n6).

The connecting portion can also have a stop with a stop surface, wherein the stop surface can be, for example, essentially perpendicular to the cone or cylinder axis or can be at an angle thereto. The stop surface is preferably embodied essentially rotationally symmetrical with respect to the cone or cylinder axis.

An implant component according to the present disclosure can comprise a metal alloy, for example a titanium-based alloy or a cobalt-based alloy. The implant component is preferably substantially made of a metal alloy, for example a titanium-based alloy or a cobalt-based alloy. In other words, the Zr coating is preferably applied to a connecting portion of an implant component essentially formed from one of the aforementioned alloys. In particular, an implant component according to the disclosure can have a CoCr cast alloy containing, for example, approximately 62-66% by weight cobalt, approximately 27-31% by weight chromium, and 4-5% by weight molybdenum. However, such an alloy can also contain small amounts of carbon, silicon, manganese, iron, and/or other accompanying elements (e.g. according to ISO 5832-4, ASTM F75). Other examples of metal alloys that can be used in an implant component are CoCr forge alloys (e.g. according to ISO 5832-12), steel alloys (e.g. according to ISO 5832-1), or titanium alloys (e.g. according to ISO 5832-3 or ISO 5832-11).

However, the implant component can also have other materials, for example a ceramic or a type of plastic, or can be substantially made from one or more of these materials. Aluminum, titanium, zirconium, and/or magnesium-based ceramics, for example, can be used as ceramic materials. Types of plastic such as UHMW-PE, PP, PEEK, or POM are also possible.

The metallic materials mentioned offer, for example, high mechanical strength combined with excellent biocompatibility properties. In addition, titanium alloys in particular are known for excellent bone tissue ingrowth behavior. The ceramic materials mentioned can have advantages in terms of fatigue strength. The above types of plastic may have advantageous slip properties, advantageous impact resistance, advantageously high ductility, and/or excellent strength properties by weight.

An implant component according to the present disclosure can be, for example, a prosthesis shaft, an intermediate piece, or a joint component, in particular a joint head. The implant component can be embodied, for example, to be used as part of a hip joint replacement prosthesis, a knee joint replacement prosthesis, an elbow joint replacement prosthesis, a shoulder joint replacement prosthesis, or a foot, hand, or finger joint replacement prosthesis.

In the case of an implant component according to the disclosure, the Zr coating can have a Zr content of at least 90 At. %, for example. The Zr coating preferably has a Zr content of at least 94 At. %, and particularly preferably at least 99.5 At. %. These figures are to be understood as proportions of substance. When the coating has a high degree of purity, for example, in the Zr proportions mentioned above, a reduction in the susceptibility to crevice corrosion and/or friction corrosion can be particularly pronounced.

In the case of an implant component according to the disclosure, the coating can be applied using a physical vapor deposition process or using an electroplating process. An electroplating process can have cost advantages over other processes. A vapor deposition method (e.g. PVD method) is characterized by a high degree of accuracy in maintaining the desired layer thickness. For example, deviations of no more than ±20% (relative to a target layer thickness) can be achieved with a PVD process. In particular, very uniform layer thicknesses can be produced with vapor deposition processes, even given complex geometries. A coating applied in a vapor deposition process also adheres to the surface of the coated workpiece significantly better than a coating applied by any other process.

According to the present disclosure, a modular endoprosthesis is also provided and has at least one, but preferably exactly one, of the implant components according to the disclosure described above. The modular endoprosthesis according to the disclosure preferably also has a second implant component having a connecting portion counterpart, wherein the connecting portion counterpart is embodied to engage with the connecting portion of the implant component according to the disclosure. In other words, the connecting portion and the connecting portion counterpart are embodied in a complementary manner. The connecting portion counterpart preferably does not have a Zr coating.

It has been shown that susceptibility to crevice corrosion and/or friction corrosion is reduced simply if the connecting portion of a first implant component of a modular endoprosthesis has a Zr coating according to the disclosure, while a connecting portion counterpart of a second implant component connected to the connecting portion does not have a Zr coating. However, in a modular endoprosthesis according to the disclosure, both the connecting portion and the connecting portion counterpart, connected to the connecting portion of a second implant component, can have a Zr coating.

In a modular endoprosthesis according to the disclosure, the first implant component can be a prosthesis shaft, for example, in which the connecting portion is a male cone, and the second implant component can be a joint component, for example, in particular a joint head, in which the connecting portion counterpart is embodied as a female cone.

In addition, the use of a Zr coating to prevent corrosion on a connecting portion of an implant component is also disclosed, wherein the coating has a thickness of 1-20 μm, preferably 1-6 μm, and particularly preferably 3-5 μm, and wherein the implant component is preferably one of the implant components described according to the disclosure above. This use has the same or comparable advantages or effects as an implant component according to the disclosure or a modular endoprosthesis according to the disclosure.

Furthermore, the use of an implant component or an endoprosthesis as a replacement hip joint in a patient with a metal allergy is also disclosed. In this context it is pointed out that different patients can exhibit different reactions to the occurrence of crevice corrosion and/or friction corrosion. For example, it is possible for a first patient to exhibit no detectable reaction to substances that are released as a result of crevice corrosion and/or friction corrosion. On the other hand, a second patient may exhibit allergic reactions to any substances released due to crevice corrosion and/or friction corrosion. Thus, the advantages of an implant component according to the disclosure or a modular endoprosthesis according to the disclosure become manifest in particular in patients in whom the allergic reactions described above can occur.

### BRIEF DESCRIPTION OF THE DRAWINGS

FIG. 1 depicts an embodiment of a modular endoprosthesis according to the present disclosure in a disassembled state;

FIG. 2 depicts the embodiment of the modular endoprosthesis from FIG. 1 in a connected state;

FIG. 3a depicts the course of the current measured during cyclic loading of an endoprosthesis having a known implant component;

FIG. 3b depicts the course of the current measured during cyclic loading of an endoprosthesis having an implant component according to the present disclosure.

### DETAILED DESCRIPTION OF PREFERRED EMBODIMENTS

The preferred embodiments described below are merely examples and are not to be considered as limiting. The same reference symbols that are listed in different figures designate identical, corresponding, or functionally similar elements.

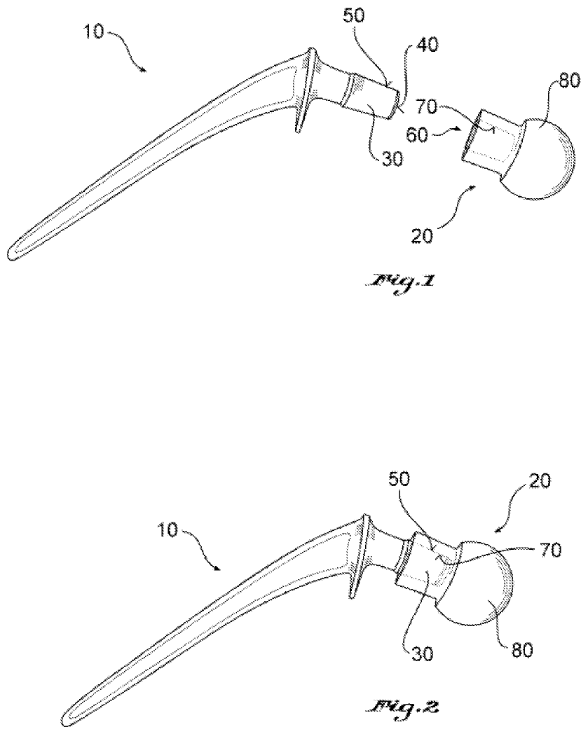

FIG. 1 depicts an embodiment of a modular endoprosthesis according to the present disclosure in an unconnected state. The modular endoprosthesis has a prosthesis shaft (10) as the first implant component and a joint head (20) as the second implant component. The prosthesis shaft (10) has a connecting portion embodied as a male cone (30). The male cone (30) has an end face (40) and an outer circumferential surface (50). The male cone (30) is coated, at least partially, with a Zr coating, the Zr coating having a layer thickness of 1-20 μm, preferably 1-6 μm.

Preferably at least the entire outer circumferential surface (50) of the male cone (30) is coated with a Zr coating. Furthermore, the end face (40) of the male cone (30) can also be coated with a Zr coating. The transition from the outer circumferential surface (50) of the male cone (30) to the end face (40) of the male cone (30) can be embodied, for example, as a chamfer or edge rounding. This transition (i.e. the chamfer or edge rounding) can also have a Zr coating according to the disclosure.

The joint head (20) has a connecting portion counterpart embodied as a female cone (60). The female cone (60) has an inner circumferential surface (70) and a bottom surface. The joint head (20) also has a joint ball (80). The joint ball is preferably embodied as a ball segment having an essentially spherical surface which acts as the joint surface of this joint component. In FIGS. 1 and 2, the female cone (60) is embodied in a connection region essentially opposing the joint ball. The connection region with the female cone (60) embodied therein projects from the side of the joint ball delimiting the ball segment. The female cone (60) can extend into the region of the ball segment. It is also possible for the connection region not to embody a projection, but rather to embody a surface that defines the ball segment and thus the joint surface. In this case the female cone (60) is formed inside the ball segment.

In the present embodiment, neither the inner circumferential surface (70) nor the bottom surface of the female cone (60) has a Zr coating. In a modification to the embodiment described above, however, the female cone (60) of the joint head (20) can also have a Zr coating, while the male cone (30) of the prosthesis shaft (10) has no Zr coating. According to a further modification, both the female cone (60) of the joint head (20) and the male cone (30) of the prosthesis shaft (10) can have a Zr coating. Preferably at least the entire outer circumferential surface (50) of the male cone (30) or the entire inner circumferential surface (70) of the female cone (60) is coated with a Zr coating.

In a variant of the embodiment described above (not shown in FIGS. 1 and 2), the female cone (60) and the male cone (30) can also be exchanged. For example, the prosthesis shaft (10) can also have a female cone, and the joint head (20) can have a male cone.

FIG. 2 depicts the above-described embodiment of the modular endoprosthesis according to the disclosure in a connected state. It can be seen from FIG. 2 that, in the connected state, the inner circumferential surface (70) of the female cone (60) is in contact with the outer circumferential surface (50) of the male cone (30). In order to avoid a double fit (which could prevent a wedging effect for the male cone (30) and the female cone (60)), it is preferred that the geometry of the male cone (30) and the geometry of the female cone (60) are matched to one another such that the end face (40) of the male cone (30) and the bottom surface of the female cone (60) are not in contact.

If the end face (40) of the male cone (30) and the bottom surface of the female cone (60) (and/or any chamfers, radii, or transition regions) are not in contact with one another, it can be assumed that no crevice corrosion and/or friction corrosion will occur on these surfaces. Thus, a Zr coating is not necessary on these surfaces. Nevertheless, a Zr coating can be provided on these surfaces. In particular, it can be advantageous for other reasons to provide a Zr coating on the end face (40) of the male cone (30), on the bottom surface of the female cone (60), and/or on any chamfers, radii, or transition regions. If a Zr coating is also provided in these regions, for example, a transition from an uncoated surface to a coated surface can be prevented there, which in turn reduces the risk of parts of the coated surface spalling (e.g. as a result of a notching effect and/or as a result of stress peaks). In addition, there is no need to mask these surfaces during the coating process (if the end face (40) of the male cone (30), the bottom surface of the female cone (60) and/or any chamfers, radii, or transition regions are also coated). This in turn results in cost advantages.

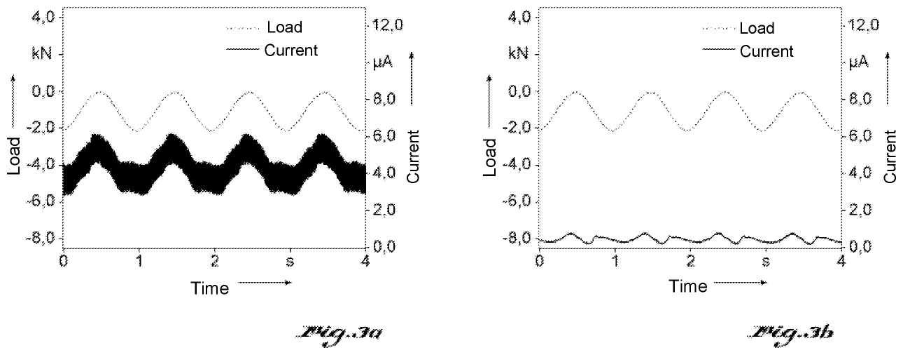

FIGS. 3a and 3b illustrate (in extracts) measured values of an experimental investigation in which a known implant component (FIG. 3a) and an implant component having a Zr coating according to the disclosure (FIG. 3b) were examined for the occurrence of crevice corrosion and/or friction corrosion. Qualitative or comparative information about the susceptibility of a connecting portion to crevice corrosion and/or friction corrosion can be obtained as an experimental investigation, for example using a measurement method according to ASTM F1875-98 (reapproved in 2014). In this method, femoral stem and head components are placed in a medium, for example a saline solution, and subjected to a cyclic load. The shaft and head components are connected by means of a connecting portion. Also placed in the medium are reference electrodes whose coating material matches the coating material of the shaft and head components to be tested. The surface area of the reference electrodes and the surface area of the (parts of the) shaft and head components added to the medium correspond to one another.

The shaft and head components as well as the reference electrodes are connected to a current measuring device that permits currents flowing (via the medium) between the shaft and head components, on the one hand, and the reference electrodes, on the other hand, to be measured. However, since the surface and the coating material of the shaft and head components and of the reference electrodes are identical, these currents are not due to a potential difference between the shaft and head components and the reference electrodes (galvanic cell/battery effect). On the contrary, the measurable current flow between the shaft and head components, on the one hand, and the reference electrodes, on the other hand, results from the fact that, as a result of the cyclical force mentioned above, parts of the passive layer (or passive layers) on the surface (or surfaces) of the connecting portion are abraded and re-form.

The average current Im over time and the average dynamic current Id can be determined from the measured values for the current flow between the shaft and head components and the reference electrodes. The average dynamic current Id is the difference between the maximum current Iₘₐₓ measured in a specific time interval and the minimum current Iₘᵢₙ (i.e., in the time interval Δt1 the following applies: I_{d,Δt1}=I_{max,Δt1}−I_{min,Δt1}).

If a lower Im value is measured for a first combination of shaft and head component than for a second combination of shaft and head component, this is considered to be indirect evidence that the first combination of shaft and head component is less susceptible to crevice corrosion and/or friction corrosion than the second combination of shaft and head component.

In particular, if a lower value Im and a lower value Id are measured for a first combination of shaft and head component than for a second combination of shaft and head component, this is considered to be indirect evidence that the first combination of shaft and head component is significantly less susceptible to crevice corrosion and/or friction corrosion than the second combination of shaft and head component.

The known implant component of FIG. 3a and the implant component having a Zr coating according to the disclosure of FIG. 3b were each connected to a further implant component having a connecting portion, in each case forming an endoprosthesis. The geometry of the endoprosthesis having the known implant component and the geometry of the endoprosthesis with the implant component with the Zr coating according to the disclosure were identical. Furthermore, identical test parameters were selected for both endoprostheses. As can be seen from FIGS. 3a and 3b, the endoprostheses were subjected to a cyclic load, the frequency of which was 1 Hz. The magnitude of the load ran periodically between 0.04 kN and 2.04 kN.

In the charts in FIG. 3a and FIG. 3b, the load (in kilonewtons) is plotted on the left-hand vertical axis, the negative sign for the load being due to its orientation (compression load). The time in seconds is entered on the respective horizontal axis of the charts. The current (in microamperes) measured between the endoprostheses and the reference electrodes during the cyclic loading of the endoprostheses is plotted on the right vertical axis in each case.

As can be seen from FIG. 3a, a current Iₘ=4.49 μA averaged over time and a mean dynamic current I_{d}=3.61 μmA were measured in a specific time interval for the endoprosthesis with the known implant component. As can be seen from FIG. 3b, a current Iₘ=0.49 μA averaged over time and a mean dynamic current I_{d}=0.68 μmA were measured in a specific time interval for the endoprosthesis with the implant component with the Zr coating according to the disclosure. Comparing the test series leads to the conclusion that the endoprosthesis having the implant component with the Zr coating according to the disclosure is less susceptible to crevice corrosion and/or friction corrosion than the endoprosthesis with the known implant component.

1. An Implant component, comprising at least one connecting portion, the connecting portion being at least partly coated with a Zr coating and the coating having a thickness of 1-20 μm.

2. The implant component of claim 1, wherein the coating has a thickness of 3-6 μm.

3. The implant component of claim 1, wherein the connecting portion comprises a female or a male cone.

4. The implant component of claim 1, wherein the connecting portion is rotationally symmetrical.

5. The implant component of claim 1, wherein the implant component comprises a titanium-based alloy or a cobalt-based alloy.

6. The implant component of claim 5, wherein the implant component comprises a CoCr alloy.

7. The implant component of claim 1, wherein the implant component comprises a prosthesis shaft, an intermediate piece, or a joint head.

8. The implant component of claim 1, in which the Zr coating has a Zr content of at least 90 At. %.

9. The implant component of claim 1, wherein the coating is applied by a physical vapor deposition process or by an electroplating process.

10. The modular endoprosthesis comprising at least one implant component of claim 1

11. The modular endoprosthesis of claim 10, further comprising a second implant component having a connecting portion counterpart, said connecting portion counterpart engageable with said connecting portion of said first implant component, wherein said connecting portion counterpart does not have any Zr coating.

12. The modular endoprosthesis of claim 11, wherein the first implant component is a prosthesis shaft having a male cone connecting portion, and second implant component is a joint head comprising a connecting portion female cone counterpart embodied as a female cone.

13. A method of preventing corrosion of an implant comprising applying a Zr coating to a connecting portion of the implant component of claim 1, said coating applied to a thickness of 1-20 μm.

14. The method of treating a patient in need of a hip replacement joint who has a metal allergy comprising: implanting the implant component of claim 1 and the implant component is a component of a hip prosthesis.

15. The method of claim 14, wherein the implant is a modular endoprosthesis.

16. The implant component of claim 8, wherein the Zr coating has a Zr content of at least 97 At. %.

17. The implant component of claim 8, wherein the Zr coating has a Zr content of at least 99.5 At. %.
